(19) United States
(12) Patent Application Publication
(10) Pub. No.: US 2019/0274728 A1
(43) Pub. Date: Sep. 12, 2019

(54) Expanding Surgical Portal

(71) Applicant: Quandary Medical, LLC, Wilmington, NC (US)

(72) Inventor: Leighton LaPierre, Thornton, CO (US)

(73) Assignee: Quandary Medical, LLC, Wilmington, NC (US)

(21) Appl. No.: 16/294,854

(22) Filed: Mar. 6, 2019

Related U.S. Application Data
(60) Provisional application No. 62/639,677, filed on Mar. 7, 2018.

(51) Int. Cl. A61B 17/34; A61B 17/02; A61B 17/70; A61M 29/00

(52) U.S. Cl. CPC A61B 17/3423; A61B 17/0293; A61B 2017/564; A61M 29/00; A61B 2017/0225; A61B 17/7074

(57) ABSTRACT

An expanding surgical dilator for access to the spine and associated method of use is presented. The method has several steps associated with utilizing the expanding surgical dilator to facilitate the placement of objects, optionally implants and/or instrumentation, through the expanding surgical dilator that exceed the expanding surgical dilator's diameter in its compressed form. The expanding surgical dilator comprises an elastic sheath that can stretch and accommodate and link blades forming the rigid bodies of the sheath.

### CROSS-REFERENCE TO RELATED APPLICATIONS

This application claims the benefit of U.S. Provisional Patent Application 62/639,677 filed on Mar. 7, 2018, which is hereby incorporated by reference in its entirety.

### BACKGROUND OF THE INVENTION

The present inventor has recognized the problem of accessing the spine through a small diameter target space without adversely impacting the surrounding nerves. Other typical prior art solutions to place objects into the body typically associated with surgical approaches, in particular objects such as surgical instrumentation or implants, require a high degree of tissue cutting. Alternatively, other prior art solutions to place objects into the spine require the placement of a portal with a fixed diameter at least as large as the diameter of each object. This approach severely limits the ability of a user to place a retractor into a small area without significantly splitting or otherwise greatly harming the tissues surrounding the portal, including in some instance's nerves, with a fixed diameter. In many cases, the risk of damage increases as the size of the portal increases. In many cases, the placement of a large number of sequential dilators, with each dilator progressively larger, risks catching, tearing or stretching the surrounding tissues increasingly as a larger diameter dilator is placed. Other solutions include articulating bladed retractor, where the expansion of the retractor occurs mechanically. In this solution, however, as the blades move an opening or split occurs between the blades exposing the tissue to objects passed through the construct. The associated problems are particularly acute in association with surgical approaches to the spine, which have a high number of sensitive tissues such as nerves and blood vessels near the approach trajectory. A more specific problem associated with the posterior oblique lateral lumbar interbody fusion approach (known in the prior art as the “OLLIF” or the “KeyLIF” approach) is that implants used in association with such approaches often must be delivered to the spine unprotected over a guide wire. The use of a guide wire poses the risk or concern of nerve damage caused by the guide wire during insertion through Kambin's triangle and into the disc space. Separately, the placement of a naked or unprotected implant over a guide wire through Kambin's Triangle could adversely impact the surrounding tissues, including the nerves. Other solutions for the problem of accessing the spine through a small diameter target space without adversely impacting the surrounding nerves may exist in the prior art. These solutions, however, have failed to meet one or more unsolved needs recognized by the inventor because of still-remaining challenges.

### BRIEF DESCRIPTION OF FIGURES

FIG. 1 depicts alternative view of expanding surgical portal in expanded state with an implant placed within in an embodiment of the invention.

FIG. 2 depicts alternative view of expanding surgical portal in expanded state with an implant placed within in an embodiment of the invention.

FIG. 3 depicts expanding surgical portal in expanded state with an implant placed within in an embodiment of the invention.

FIG. 4 depicts oval shaped expanding surgical portal with alternatively configured distal protrusion in an embodiment of the invention.

FIG. 5 depicts exploded view of embodiment featuring related sequential dilators in an embodiment of the invention.

FIG. 6 depicts alternative view of embodiment featuring implant entering the proximal flare in an embodiment of the invention.

FIG. 7 depicts view of embodiment featuring implant entering the proximal flare in an embodiment of the invention.

FIG. 8 depicts an embodiment of the invention where the elastic sheath contains a channel for a fixation pin.

FIGS. 9-16 depict various embodiments of the invention in various configurations.

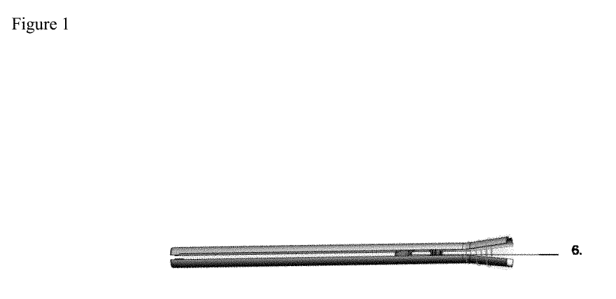

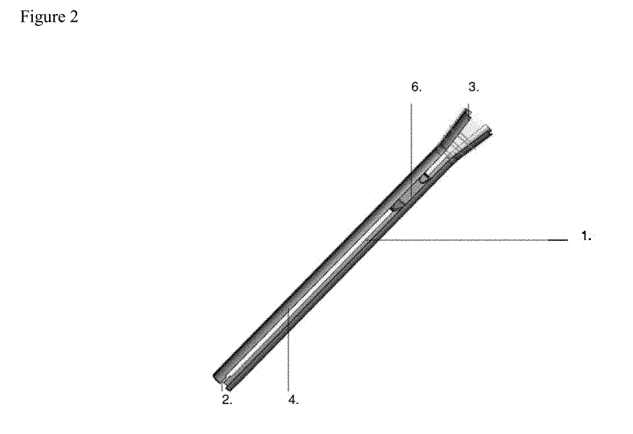

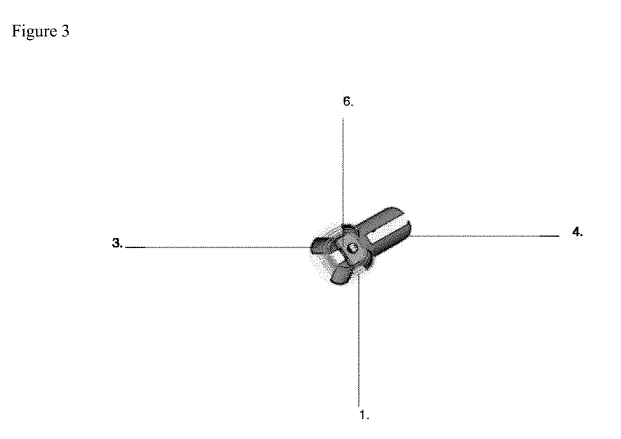

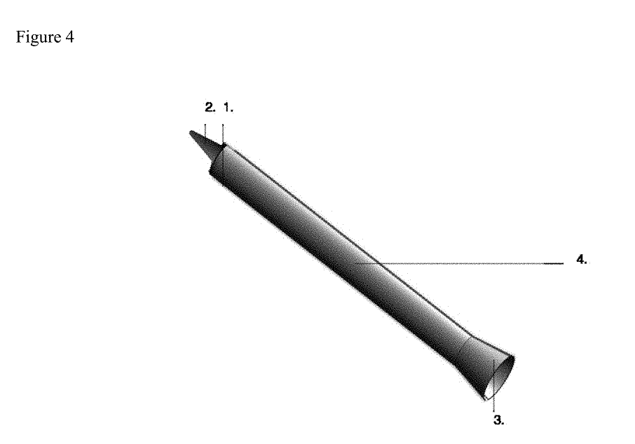

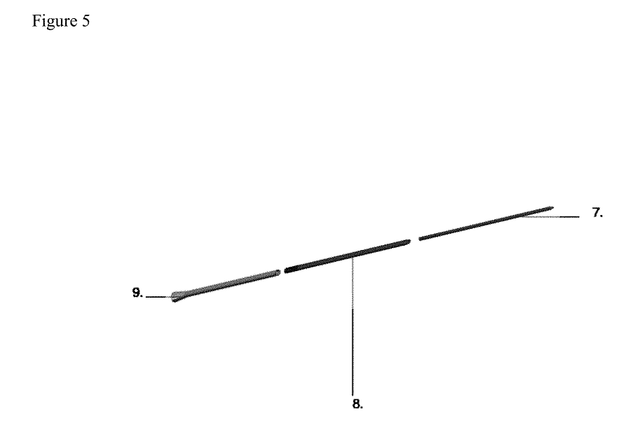

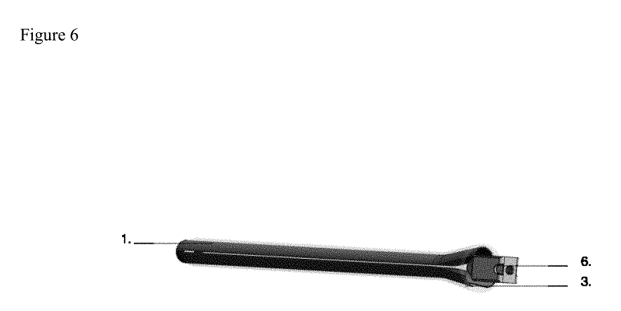

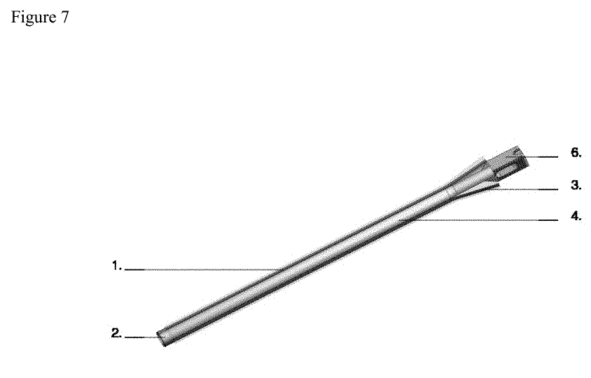

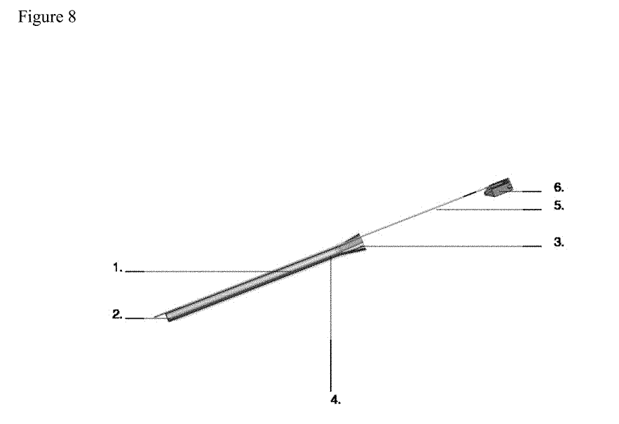

### BRIEF DESCRIPTION OF NUMERICAL REFERENCES IN FIGS.

1. Elastic Sheath in an embodiment of the invention.

2. Distal Protrusion in an embodiment of the invention.

3. Proximal Flare in an embodiment of the invention.

4. Inner Blade in an embodiment of the invention.

5. Fixation Pin in an embodiment of the invention.

6. Implant in an embodiment of the invention.

7. First Sequential Dilator in an embodiment of the invention.

8. Second Sequential Dilator in an embodiment of the invention.

9. Expanding Surgical Portal Configured To Slide Over Surgical Dilator in an embodiment of the invention.

### LEGEND OF FEATURES

1. Elastic Sheath

2. Distal Protrusion

3. Proximal Flare

4. Inner Blade

5. Fixation Pin

6. Implant

7. First Sequential Dilator

8. Second Sequential Dilator

9. Expanding Surgical Portal Configured To Slide Over Surgical Dilator

### DESCRIPTION OF THE INVENTION

The preferred embodiment of the present invention is described as an Expanding Surgical Portal. In the preferred embodiments and methods of use, the Expanding Surgical Portal solves the problems associated with delivering a substantially square or rectangular spinal cage through muscle and nerve tissue without protection. In the preferred embodiment, the Expanding Surgical Portal comprises a radially expandable tube. In an embodiment, the Expanding Surgical Portal allows an implant with a cross section of varying sizes and shapes to be delivered to a targeted region within the body, such as a targeted area of the spine, in a protected manner. Embodiments of the Expanding Surgical Portal allow for a mass having a diameter of larger than 9 millimeters to pass through a small 9 millimeter internal diameter access portal while reducing or eliminating the potential for damage to soft tissue and/or nerves during the act of inserting the mass. In varying embodiments, the mass optionally comprises a surgical implant or surgical instrumentation. In the preferred embodiment, the masses intended to be passed through the Expanding Surgical Portal include implants and instrumentation associated with spine surgery. Generally, the inventor intends for the Expanding Surgical Portal to function as an access portal for use during surgery that gently expands after placement to accommodate an object passed through it that has a cross section larger than the diametric cross section of the Expanding Surgical Portal in its compressed form without adversely impacting the surrounding tissues. In varying embodiments, the invention may incorporate any subset of, or all of, the following components: (1.) Elastic Sheath, (2.) Distal Protrusion, (3.) Proximal Flare, (4.) Inner Blade, (5.) Fixation Pin, (6.) Implant, (7.) First Sequential Dilator, (8.) Second Sequential Dilator, (9.) Expanding Surgical Portal Configured To Slide Over Surgical Dilator. In the intended use of the preferred embodiment of the invention, a user (generally a surgeon) places the Expanding Surgical Portal after creating an incision and identifying an approach trajectory to a targeted anatomical structure within a patient's body. In an embodiment of the invention, the targeted anatomical structure is a specific area adjacent to or within the spine. In an embodiment of the invention, the Expanding Surgical Portal is placed generally along the approach trajectory to facilitate the placement of objects which have a diameter larger than the smallest diameter of the Expanding Surgical Portal in its collapsed form through the Expanding Surgical Portal in a manner that only gently and transiently places pressure on the anatomical structures external to the Expanding Surgical Portal as the objects move through the Expanding Surgical Portal, thereby expanding the Expanding Surgical Portal from its collapsed form to its expanded form. In an embodiment of the invention, in the intended use, the user (or the surgeon) places the Distal Protrusion to a targeted point at or near the distal end of the approach trajectory.

An embodiment of the invention incorporates at least two Inner Blades 4. The preferred embodiment of the Inner Blades 4 comprises the following dimensions: approximately 20 centimeters in length, and curved such that the tubular form of the Inner Blades 4 when placed together have a 9 millimeter inner diameter when placed together in the compressed form. The preferred embodiment of the Inner Blade 4 incorporate stainless steel as the sole or primary element in its composition. An Inner Blade 4 in an embodiment of the invention is described as the structural rigid component along the interior surface of the expandable surgical portal.

An embodiment of the invention incorporates a Distal Protrusion 2, as a sub-component of either or both of one or more of the Inner Blades 4 and/or the Elastic Sheath 1. The preferred embodiment of the Distal Protrusion 2 comprises the following dimensions: 10 millimeters outer diameter in the compressed form and extending from the Elastic Sheath by approximately 5 millimeters. In an alternative embodiment of the invention, the Distal Protrusion forms a less rigid portion of the Elastic Sheath 1 and extends distally beyond a more rigid component of the Elastic Sheath 1 approximately 5 millimeters. The preferred embodiment of the Distal Protrusion 2 incorporates stainless steel in its composition. An alternative embodiment of the Distal Protrusion 2 incorporates medical grade silicone as the primary material in its composition. A Distal Protrusion 2 in the preferred embodiment of the invention is more precisely described as the portion of one or more of the Inner Blades 4 that extends distally beyond the Elastic Sheath 1.

An embodiment of the invention incorporates an Elastic Sheath 1. The preferred embodiment of the Elastic Sheath 1 has a diameter slightly larger than the diameter of Inner Blades 4 placed together in a compressed form to comprise a substantially tubular shape. An embodiment of the Elastic Sheath 1 has a thickness of no more than 2 millimeters.

The Elastic Sheath 1 in an embodiment of the invention is described as an expandable thin and elastic material forming the outer barrier of the Expanding Surgical Portal. In an embodiment, the Elastic Sheath 1 is attached to the exterior of each of the Inner Blades 4. In an alternate embodiment the blades or formation of blades is inserted into the Elastic Sheath by the user. In an alternative embodiment of the invention, the Elastic Sheath 1 obviates the need for Inner Blades 4 by incorporating properties and dimensions such that the separate Inner Blades 4 are unnecessary. The preferred embodiment of the Elastic Sheath 1 incorporates medical grade silicone in its composition. In an embodiment, the Elastic Sheath 1 comprises materials that meet the biocompatibility requirements of the ISO 10993 standard, which is incorporated by reference herein. The present inventor has noted the importance of the Elastic Shape 1 comprising a durable material that is resistant to tearing or failure, especially when in the expanded form. In an embodiment of the invention, the medical grade silicone comprising the primary material of the elastic sheath consists of DOW Silastic Q7-4780 silicone. In an embodiment of the invention, the Elastic Sheath 1 incorporates a flexible distal tip to contour to the vertebral body. In an embodiment, the flexible distal tip of the Elastic Sheath 1 comprises medical grade silicone of a different composition than the rest of the Elastic Sheath 1. In an embodiment of the invention, the flexible distal tip of the Elastic Sheath 1 consists primarily of Wacker Elasosil LR 3071/40 silicone. The Elastic Sheath 1 in an embodiment of the invention is also described as an apparatus that can stretch beyond its initial diameter to an expanded form and return to the collapsed form. In embodiments of the invention, the Elastic Sheath 1 functions to contain two or more Inner Blades 4 in both the collapsed form and the expanded form. In the expanded form, the portion of the Elastic Sheath 1 between the Inner Blades 4 stretches to increase the distance between the Inner Blades 4. This action thereby allows for increased diameter of the Expanding Surgical Portal as an object with a cross section larger than the diametric cross section of the Expanding Surgical Portal in its collapsed shape passes through the Expanding Surgical Portal. The present inventor has also noted the importance of an embodiment of the Expanding Surgical Portal having smooth exterior surface. In an embodiment of the invention, the smooth exterior surface of the Elastic Sheath 1 forming the outer barrier of the Expanding Surgical Portal is intended to prevent trauma to the tissues and structures immediately exterior to the Expanding Surgical Portal during intended use. The Elastic Sheath 1 in an embodiment of the invention incorporates a hydrophilic outer surface. The present inventor has noted many advantages of the hydrophilic outer surface into embodiments of the invention, such as in the outer most surface of the Elastic Sheath 1. In embodiments of the invention, the hydrophilic outer surface is applied to the Elastic Sheath 1 utilizing techniques of hydrophilic coating as known by those skilled in the art. Such advantages of incorporating a hydrophilic surface into the outer most barrier of the invention include the reduction of friction during insertion into the body, as the hydrophilic surface would assist in creating a wettable, lubricious surface suitable for biologic interactions. Other advantages include the added ease of sterilization of the Expanding Surgical Portal prior to intended use.

An embodiment of the invention incorporates a Proximal Flare 3. In in an embodiment of the invention, the Proximal Flare 3 is described as a portion of the Expanding Surgical Portal near its proximal end having an enlarged diameter which gradually reduces to the diameter of the Inner Blades 4 in their collapsed form to facilitate the initial placement of larger bodies within the Expanding Surgical Portal. The preferred embodiment of the Proximal Flare 3 comprises an open-tipped cone, having a diameter of approximately 20 millimeters at the most proximal portion, and having a diameter similar to the diameter of the Inner Blades 4 in their compressed form or of a diameter similar to the diameter of the Elastic Sheath 1 at its distal portion. The preferred embodiment of the Proximal Flare 3, incorporates the proximal ends of the Inner Blades and elastic sheath in its composition.

An embodiment of the invention incorporates a Fixation Pin 5. A Fixation Pin 5 in an embodiment of the invention is described as an elongate body designed to slide through a corresponding aperture within the Expanding Surgical Portal. In an embodiment of the invention, the Fixation Pin 5 is designed to traverse an aperture contained within the Elastic Sheath 1 of the Expanding Surgical Portal. During intended use, an embodiment of the Fixation Pin 5, after traversing an aperture within the Expanding Surgical Portal, is intended to engage with an anatomic structure to hold a portion of the Expanding Surgical Portal in a fixed position. In an embodiment of the invention, during intended use the present inventor has recognized the benefits of locking the portion of the Expanding Surgical Portal nearest the most sensitive external tissues, such as the nerves, into a rigid anatomic structure, such as the bony tissue of the spine, and thereby expanding the portions of the Expanding Surgical Portal lacking direct interaction with a Fixation Pin 5 away from the portion of the Expanding Surgical Portal having direct interaction with a Fixation Pin 5 placed as intended, the latter portion that has direct interaction with a Fixation Pin 5 remaining in a stationary, fixed position. The preferred embodiment of the Fixation Pin 5 comprises a length longer than the length of Elastic Sheath 1 with a diameter of 4 millimeters or less. The preferred embodiment of the Fixation Pin 5, incorporates stainless steel in its composition.

An embodiment of the invention incorporates an Implant 6. An As used herein, the Implant 6 in an embodiment of the invention is described as an example of any structure placed through the Expandable Surgical Portal.

In the preferred method of use, as the Implant 6 is placed into the Expanding Surgical Portal, first by placement into the Proximal Flare 3, the Implant 6 contacts the inner surface of the Expanding Surgical Portal. In an embodiment, the inner surface of the Expanding Surgical Portal comprises the inner surface of the Inner Blades 4. As the Implant 6 is advanced through the Proximal Flare 3 and into the interior of the Inner Blades 4, it places force upon the inner surface of the Inner Blades 4, causing the Inner Blades 4 to separate from each other. As the Inner Blades 4 separate from each other, the Elastic Sheath 1 stretches to expand, thereby increasing the diameter of the Expanding Surgical Portal as the Implant 6 advances through. During this process, in the preferred embodiment, the Elastic Sheath 1 maintains the cohesion of the Expanding Surgical Portal as it serves as the connective body between at least the Inner Blades 4.

An embodiment of the invention incorporates a First Sequential Dilator 7. The First Sequential Dilator 7 has a small diameter designed to define the pathway for the Expanding Surgical Portal to follow. In embodiments of the invention, the Expanding Surgical Portal is designed to slide over the First Sequential Dilator 7 during intended use. In alternative embodiments, an intermediary Second Sequential Dilator 8 is slid over the First Sequential Dilator 7 prior to placement of the Expanding Surgical Dilator over the Second Sequential Dilator 8.

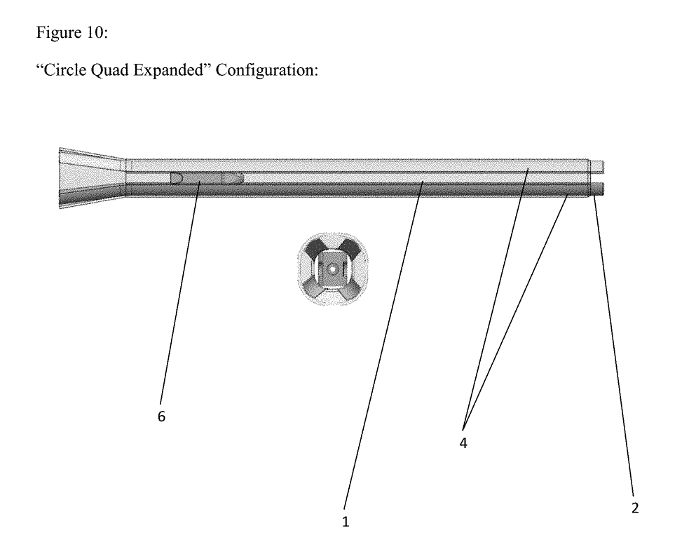

The following depicts an exemplary series of steps associated with an intended use of an embodiment of the Expandable Surgical Dilator in association with various approaches, implants and instrumentation associated with spine surgery, including the oblique lateral lumbar interbody fusion (OLLIF) spinal procedure and the associated instrumentation and implants, for instance those disclosed in United States Patent Application 20110230965A1 filed on Feb. 6, 2011 and U.S. patent application Ser. No. 13/294,544 filed on Nov. 11, 2011, which are hereby incorporated by reference in their entirety:
1. Find docking point in the spine using probe and neuromonitoring, as known by those skilled in the art;
2. Dilate up using First Sequential Dilator 7 and optionally Second Sequential Dilator 8;
3. Insert Expanding Surgical Portal in its collapsed form over outermost dilator to a point where the Distal Protrusion 2 is docked within a disc space;
4. Perform disc prep and bone grafting through Expanding Surgical Portal in its collapsed form;
5. Insert implant (such as a cannulated or non-cannulated PEEK or Titanium interbody Implant 6 associated with spinal fusion, known by those skilled in the art) through Expanding Surgical Portal; (In alternative embodiments, the Implant could either be a solid singular structure or expandable in nature. In the case where the Implant is expandable in nature, the present inventors contemplate that during this step the Implant would be inserted in its collapsed state.)
6. The Implant 6 will self distract the Expanding Surgical Portal as it travels along the Expanding Surgical Portal's longitudinal axis, as depicted by FIG. 10;
a. The Expanding Surgical Portal will only expand temporarily while Implant 6 is passing through and will collapse back down after Implant 6 delivery to the disc space;
7. Remove Expanding Surgical Portal.

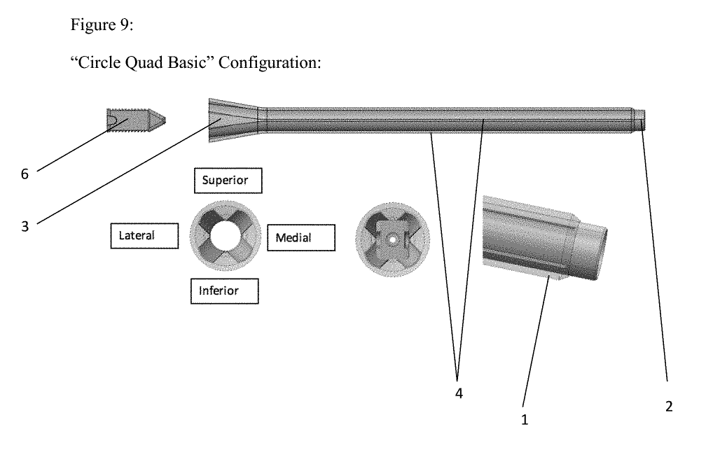

The following describes the embodiments of the invention depicted as various configurations in FIGS. 9-16. FIG. 9 depicts the “Circle Quad Basic” configuration as an embodiment of the invention. In this configuration, an embodiment of the Expandable Surgical Portal comprises of four Inner Blades 4 curved in the longitudinal direction arranged in a circular tubular pattern. In such embodiment, each Inner Blade 4 is cut in a radial 80 degree arc with the inner radius preferably equaling 4.5 millimeters. In such embodiment, the proximal end of the blade is bent outward to form a ramp or funnel for gradual insertion (forming part of the Proximal Flare 3). The present inventor has recognized that such outward bending assists with ease of insertion and guiding of a surgical instrument, implant, or inserter during intended use. In such embodiment, the assembly comprising four Inner Blades 4 (though the assembly could alternatively comprise two or three Inner Blades 4 as shown in additional embodiments below) are surrounded by a pliable/stretchable material, preferably comprising a medical grade silicone, described herein as the Elastic Sheath 1. The distal end of one or more of the Inner Blades 4 in such embodiment is cut in a radial 90 degree arc such that the four blades together make a complete circular perimeter at the distal tip of the tube, comprising the Distal Protrusion 2. The complete circular perimeter partially extends beyond the opening of the outer silicone enclosure, such extension referred to herein as the Distal Protrusion 2, and partially is contained within the lumen of the Elastic Sheath 1. The exposed metal tips on the distal end comprising the Distal Protrusion 2 are intended to be secured within a disc space of a spine to provide a safe working channel for disc prep instrumentation to enter into a disc space of a spine. The present inventor recognizes that this also allows a smooth transition when inserting an Implant 6 into the disc space. The metal blades act as a graft slide, disc shim, and/or shoe horn type transition from outside of the spine to the inside of the interbody space. The present inventor recognizes that in an embodiment the metal blades comprising the Inner Blades 4 could either be molded directly into the silicone enclosure or assembles and secured into place after molding of the silicone with a medical grade adhesive or silicone adhesive. In an embodiment, the non-stretched inner diameter of the Expanding Surgical Portal depicted in FIG. 9 is 9 millimeters. The Elastic Sheath 1 can allow radial expansion to accommodate an implant that is 10 millimeters to 11 millimeters wide by up to 16 millimeters tall, as depicted by FIG. 10. In an intended use of an embodiment, the corners or edges of the Implant 6 along the axis of insertion would be oriented and centered in the concave curvature of each Inner Blade 4. In such embodiment, therefore, the four silicone “gaps” comprising portions of the Elastic Sheath 1 between each of the four Inner Blades 4 would be oriented superior, medial, inferior, and lateral respectively to the spine.

FIG. 10 depicts a similar embodiment to the Circle Quad Basic configuration shown in FIG. 9, but expanded to show a 10 millimeter wide by 11 millimeter tall Implant 6 passing through.

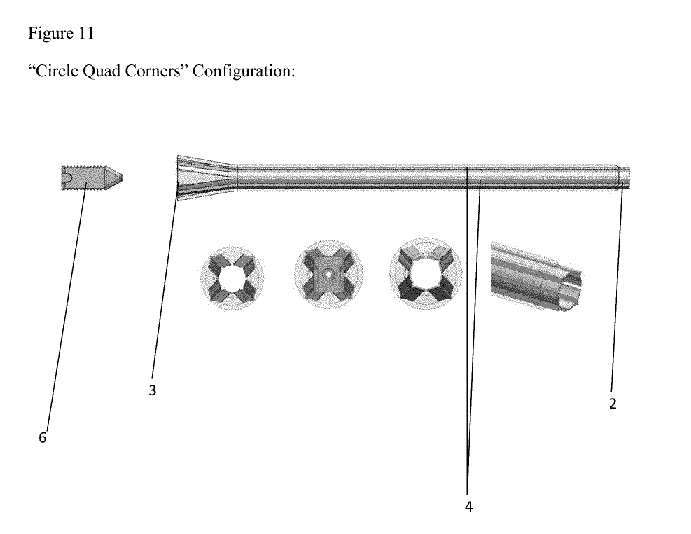

FIG. 11 depicts an embodiment configured as the “Circle Quad Corners” configuration, which is similar to the “Circle Quad Basic” configuration described above but with formed corners in each of the four Inner Blades 4 to help guide and control the rotation of the Implant 6 during insertion.

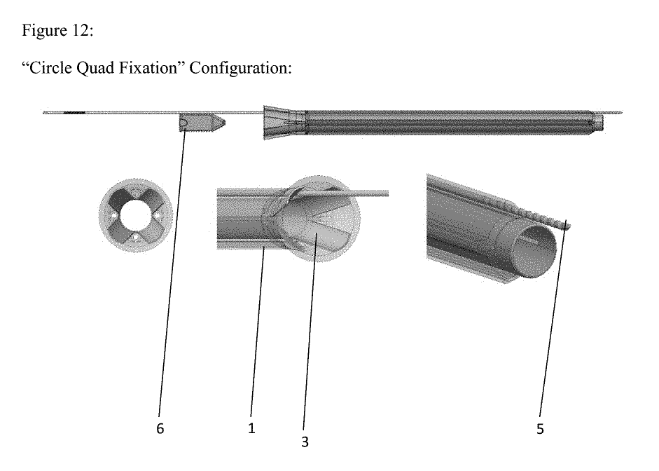

FIG. 12 depicts an embodiment configured as the “Circle Quad Fixation” configuration, which is similar to the “Circle Quad Basic” configuration described above but with an aperture for a Fixation Pin 5 or neuromonitoring channels molded or formed into the Elastic Sheath 1. In varying embodiments, the Expanding Surgical Portal optionally incorporates one to four (or more) channels around the outer diameter (or within the outer diameter but this is not shown) of the Elastic Sheath 1. These channels would allow a Fixation Pin 5 (as shown in FIG. 12) to pass through and anchor the expanding tube assembly into bone, during the preferred method of use said bone consisting of a vertebral body. Alternatively, in embodiments of the invention and associated methods of use a mono polar neuromonitoring probe as known by those skilled in the art could also be placed down one or more of these channels to measure proximity to an exiting nerve root.

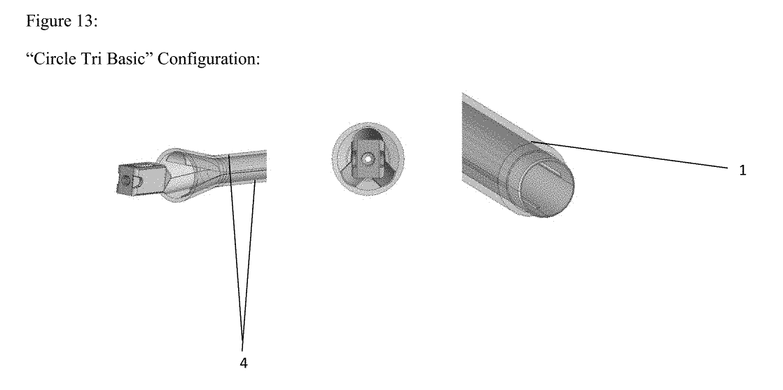

FIG. 13 depicts an embodiment of the invention comprising the “Circle Tri Basic” configuration, which is similar to the “Circle Quad Basic” configuration described above, but with one Inner Blade 4 of a size and shape more closely resembling that a semi-circular cross-sectional form (for instance, with a 170 degree radial cut), and having two other Inner Blades 4 more closely resembling a quarter circular cross-sectional form (for instance, with the same 80 degree radial cut as in the “Circle Quad Basic” configuration described above). In such embodiment, the three Inner Blades 4 of different radial cuts together make up an almost complete circular cross-section enclosed by an Elastic Sheath 1.

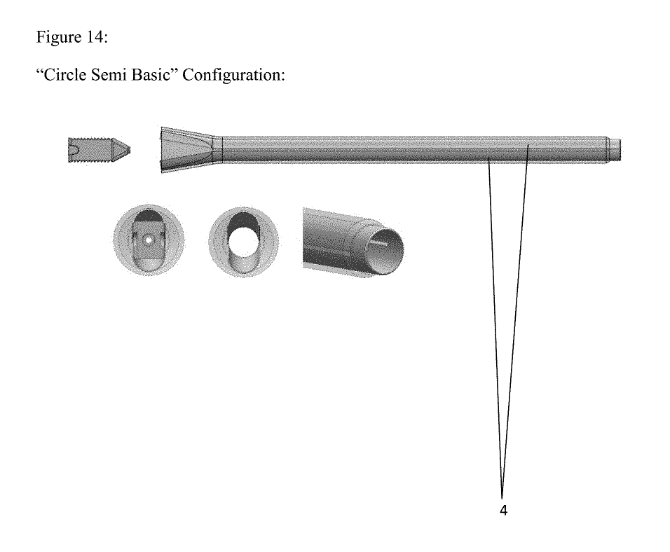

FIG. 14 depicts a “Circle Semi Basic” configuration embodiment, which is similar to the “Circle Quad Basic” configuration described above, but wherein two Inner Blades 4 each more closely resemble a semi-circular cross-sectional form (for instance, with a 170 degree radial cut on both Inner Blades 4).

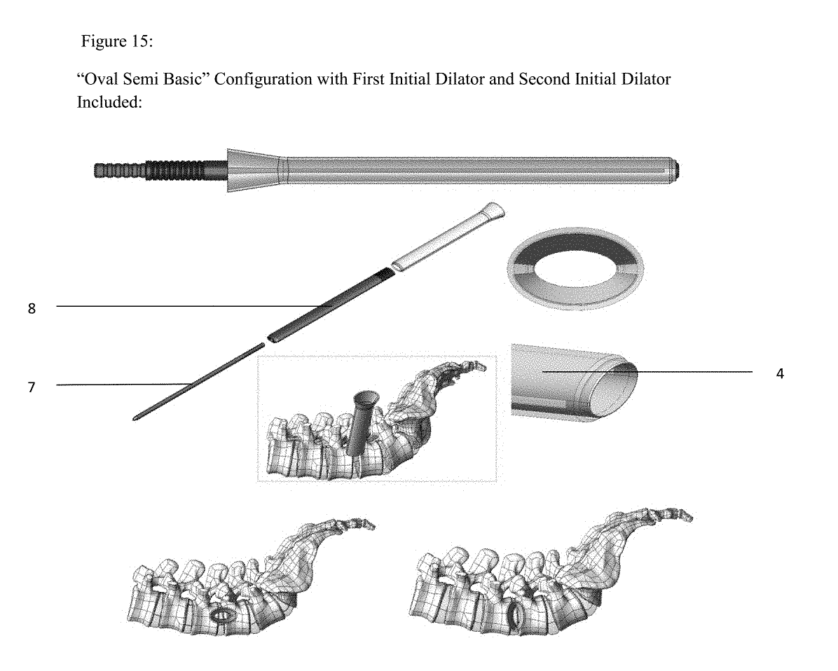

FIG. 15 depicts a “Oval Semi Basic” configuration embodiment, which is similar to the “Circle Semi Basic” configuration described above, but wherein the longitudinal cross-sectional shape of the Expanding Surgical Portal is shaped more like an ellipse or oval. Shown in this embodiment is a series of two dilators used to facilitate insertion. The First Sequential Dilator 7 has a circular cross section and is approximately 6.5 mm in diameter. The Second Sequential Dilator 8 has an elliptical cross section (could be a slot or grossly rectangular) where the major axis is approximately 20 millimeters and the minor axis is approximately 9 millimeters. In this embodiment the expansion of the Expanding Surgical Portal after placement over the Second Sequential Dilator 8 and passage of the Implant 6 through the Expanding Surgical Portal would primarily occur in the direction of the minor axis. In such embodiment, an Implant 6 that fits the width of the major axis would be placed down the Expanding Surgical Portal and the height of the cage would split the two Inner Blades 4 in the form of semi oval blades apart. In such embodiment, the oval-shaped Expanding Surgical Portal could be positioned in line (parallel) with the joint or disc space or perpendicular to the disc space when viewing the spine in the lateral or sagittal plane.

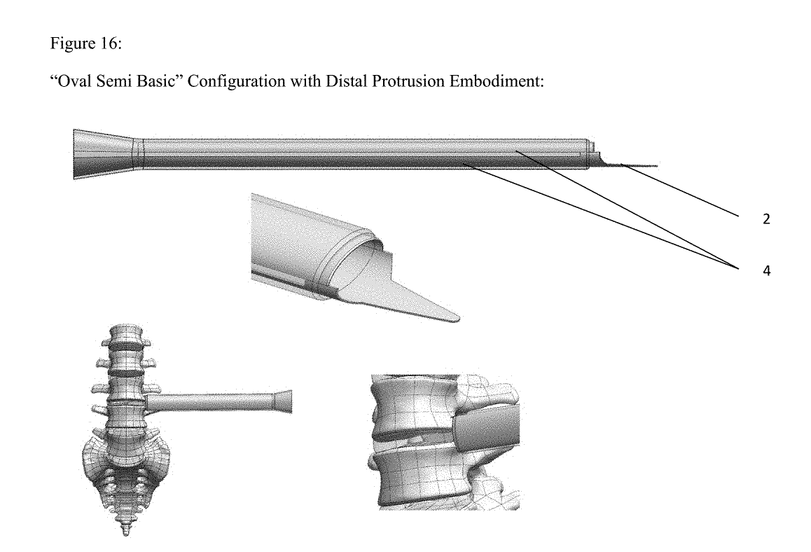

FIG. 16 depicts an “Oval Semi Basic” configuration embodiment, which is similar to the Oval Semi Basic, but wherein one Inner Blade 4 additionally has an integrated disc/bone shim to dock into the disc or bone, comprising a portion of the Distal Protrusion 2. In an alternate embodiment (not shown) the disc/bone shim could be a separate component to one of the oval semi blades and could be independently advanced down the Inner Blade 4 in a track or slot running along the length of such Inner Blade 4 to be delivered to the disc/joint or bone.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art. The term “proximal” refers to the area or point nearest the user (typically a surgeon) during intended use, and the term “distal” refers to the area or point farthest away from the user during intended use. The terms “coupled” and “linked” as used herein is defined as connected, although not necessarily directly and not necessarily mechanically. A device or structure that is “configured” in a certain way is configured in at least that way, but may also be configured in ways that are not listed. Also, the sequence of steps in a flow diagram or elements in the claims, even when preceded by a letter does not imply or require that sequence.

I claim:

1. A method of utilizing an expanding surgical dilator to accomplish access to the spine, comprising:
Finding a docking point in the spine;
Dilating using a first sequential dilator and optionally a second sequential dilator; and
Inserting the expanding surgical portal in its collapsed form over the outermost of the first sequential dilator and second sequential dilator to a point where a distal protrusion of the expanding surgical dilator is docked.

2. The method of claim 1, further comprising:
Performing disc preparation by placing surgical instruments through the expanding surgical portal.

3. The method of claim 1, further comprising:
Placing bone grafting by utilizing surgical instruments through the expanding surgical portal.

4. The method of claim 1, further comprising:
Inserting an implant through the expanding surgical portal.

5. The method of claim 1, further comprising:
Self-distracting the expanding surgical portal by the placement of at least one objects through the expanding surgical portal as the at least one object travels along the expanding surgical portal's longitudinal axis.

6. The method of claim 1, further comprising:
Collapsing the expanding surgical portal following delivery of an implant into a portion of the spine.

7. The method of claim 1, further comprising:
Removing the expanding surgical portal while in its collapsed form.

8. An expanding surgical portal, comprising:
An elastic sheath capable of expansion,
At least two inner blades, and
Each of the inner blades cohesively linked together by the elastic sheath as the connective body of the expanding surgical portal.

9. The expanding surgical portal of claim 8, the elastic sheath comprising a smooth outer surface.

10. The expanding surgical portal of claim 8, the elastic sheath consisting of a hydrophilic material.

11. The expanding surgical portal of claim 8, the elastic sheath further comprising a flare near its proximal end to create an opening of a greater diameter than the diameter of the remainder of the expanding surgical portal in its collapsed form.

12. The expanding surgical portal of claim 8 configured to expand to accommodate an implant having a dimension greater than the diameter of the main aspect of the longitudinal member of the expanding surgical portal in its collapsed form.

13. The expanding surgical portal of claim 8, the expanding surgical portal further comprising at least one channel configured to accommodate a fixation pin.

14. The expanding surgical portal of claim 8 further comprising a distal protrusion.

15. The expanding surgical portal of claim 8, comprising four inner blades featuring a circular cross section when placed into a collapsed form.
